(19) United States
(12) Patent Application Publication
(10) Pub. No.: US 2018/0161383 A1
(43) Pub. Date: Jun. 14, 2018

(54) NATURAL PHARMACEUTICAL COMPOSITION FOR TREATING MENOPAUSAL MENTAL DEPRESSION AND ATYPICAL DEPRESSIVE DISORDER AND APPLICATION THEREOF

(71) Applicant: SHAANXI TIANKUI BIOMEDICINE TECHNOLOGY LIMITED COMPANY, Xi'an (CN)

(72) Inventors: Xiazhen WEI, Xi'an (CN); Lanlan SONG, Xi'an (CN); Shaohua HAN, Xi'an (CN)

(21) Appl. No.: 15/870,581

(22) Filed: Jan. 12, 2018

Related U.S. Application Data
(63) Continuation of application No. PCT/CN2016/089822, filed on Jul. 12, 2016.

(30) Foreign Application Priority Data
Jul. 14, 2015 (CN) 201510412564.0

(51) Int. Cl. A61K 36/258; A61K 36/72; A61K 9/00; A61K 9/48; A61P 25/24; A61P 5/08; A61P 5/10

(52) U.S. Cl. CPC A61K 36/258; A61K 36/72; A61K 9/0053; A61P 5/10; A61P 25/24; A61P 5/08; A61K 9/48

(57) ABSTRACT

The present disclosure discloses a pharmaceutical composition for treating menopausal mental depression and atypical depressive disorder and an application thereof. The composition is composed of the following components in percentage by mass: 70%-97.5% of sea buckthorn pulp oil and 2.5%-30% of total panax notoginseng saponins from stem and leave. The application is use of the pharmaceutical composition for preparing drugs and/or health care products used for treating menopause, the menopausal mental depression or the atypical depressive disorder.

### TECHNICAL FIELD

The present disclosure belongs to the technical field of dose-effect action of natural drug formulas, and particularly relates to a natural pharmaceutical composition for treating menopausal mental depression and atypical depressive disorder and an application thereof.

### BACKGROUND

Menopausal mental depression is a type of neurotic disorders with psychological and physiological symptoms such as mental excitation, dispirited emotion, annoyance and depression caused by endocrine disorder, sexual organ function decline (ovarian failure or senescence). An age of first pathogenesis of the menopausal depression is menopause (55-60 for men and about 45-55 for women), and the menopausal depression is manifested as syndromes caused by factors such as anxiety, tension, melancholy and the like. Common clinical symptoms include restlessness, stress fear, bewilderment in case of slight disturbance, black mood, pessimism, endlessly weep, self-accusation, subjective assumption, suspicion of others, or suspicion of suffering some diseases, particularly “cancerophobia”, and even behaviors of autolesion, suicide and the like. In addition, the symptoms are often accompanied with irregular menstruation, sexual hypoactivity, or other symptoms such as perspiration, sensation of chill, emaciation, weakness and the like. At present, in clinical treatment of the menopausal mental depression, hormone drugs are often used for performing replacement therapy. However, as reported by the States National Institutes of Health, a risk index of estrogen therapy in aspects of angiocarpy, pulmonary embolism, tumors and the like is greater than a benefit index. Significant side effects of the estrogen therapy are clinically apparent.

Most of clinical severe patients suffering from the menopausal mental depression are often treated by monoamine oxidase inhibitors such as phenelzine, isocarboxazide, rolicyprine, moclobemide and other drugs. These drugs are particularly effective on treatment of atypical depressive disorder, and have a history in clinical treatment. The drugs have wide pharmacologic actions and have interactions with many drugs, while some interactions may produce serious consequences, causing wide hepatic enzyme inhibition effects, inhibiting normal metabolisms of monoamines, causing elevation of blood pressure, producing high hepatotoxicity, and even causing severe hypertensive crisis. It is pointed out that, the monoamine oxidase inhibitors (MAOI) are most suitable for typical depression accompanied with mental retardation. Linvingston et al. point out that the monoamine oxidase inhibitors (MAOI) have certain effects on panic and agoraphobia of the atypical depression and can be applied to hypochondriasis and irritability.

The following non-tricyclic drugs are often applied to severe menopausal mental depression: paroxetine, trazodone, mianserin, maprotiline, fluoxetine, sertraline, citalopram, venlafaxine, bupropion acetone and the like. These drugs can be used for treating generalized anxiety disorder, insomnia, irritable bowel syndrome and other diseases and are consistent with the above typical symptoms of the menopausal mental depression. These drugs have common side effects in clinical treatment as follows: weakness, constipation, dizziness, thirst, headache, blurred vision, hydrosis, insomnia, sexual dysfunction, fremitus, frequent micturition or retention of urine, nausea and emesis. Accidental symptoms include anxiety, appetite changes (increase or decrease), palpitation, paresthesia, taste loss, abnormal liver function, body weight change (increase or decrease), myalgia, myasthenia, orthostatic hypotension, angioneurotic edema, hyponatremia and urticaria. Extrapyramidal system reactions such as cathisophobia, low muscular tension, discordant muscular tension and the like may be further caused. If the non-tricyclic drug of paroxetine is suddenly not used, withdrawal syndromes may be produced. Mental depression symptoms before medication such as sleep disorders, excitement, anxiety, headache and the like may reoccur.

### SUMMARY

In order to overcome defects in the prior art, a purpose of the present disclosure is to provide a natural pharmaceutical composition for treating menopausal mental depression and atypical depressive disorder and an application thereof. The natural pharmaceutical composition of sea buckthorn pulp oil and total panax notoginseng saponins from stem and leave is appropriate in compatibility, safe in dose, fast in remarkable effects and obvious in curative effects and can effectively solve problems that the existing drugs have side effects and poor curative effects in treating the menopausal mental depression and atypical depressive disorder.

The present disclosure is realized through the following technical solutions.

A natural pharmaceutical composition for treating mental depression disclosed in the present disclosure consists of the following components in percentage by mass: 70%-97.5% of sea buckthorn pulp oil and 2.5%-30% of total panax notoginseng saponins from stem and leave.

The composition consists of the following components in percentage by mass: 90% of the sea buckthorn pulp oil and 10% of the total panax notoginseng saponins from stem and leave.

The natural pharmaceutical composition is compounded by using the sea buckthorn pulp oil and the total panax notoginseng saponins from stem and leave as active ingredients and can be prepared into soft capsules or oral liquid preparations, wherein the sea buckthorn pulp oil is extracted from fresh sea buckthorn fruits without seeds, and the total panax notoginseng saponins from stem and leave is extracted from leaves of panax notoginseng.

For pharmacodynamic content, a mass ratio of palmitic acid to hexadecenoic acid contained in the sea buckthorn pulp oil is (1.02-1.20):1; and a mass ratio of ginsenoside Rb3 to ginsenoside Rb1 contained in the total panax notoginseng saponins from stem and leave is 1:(0.16-0.20).

The present disclosure further discloses an application of the above natural pharmaceutical composition in preparation of a drug and/or a health care product for treating menopause, the menopausal mental depression and/or the atypical depressive disorder.

The drug and/or the health care product is a drug and/or health care product for regulating a content ratio of marker molecules in serum of menopause, the menopausal mental depression or the atypical depressive disorder.

The drug and/or the health care product is a drug and/or health care product for increasing a content ratio of cAMP/cGMP in the serum of the menopause, the menopausal mental depression or the atypical depressive disorder.

The drug and/or the health care product is a drug and/or health care product for decreasing the content of follicle-stimulating hormones and increasing the content of oxytocin.

The drug and/or the health care product is a drug and/or health care product for increasing the content of dehydroepiandrosterone and decreasing a ratio of dihydrotestosterone. The drug and/or the health care product is a drug and/or health care product for respectively increasing the contents of de-estradiol and dihydrotestosterone.

The drug and/or the health care product is a drug and/or health care product for increasing a content ratio of dopamine and serotonin and increasing a content ratio of noradrenaline. The drug and/or the health care product is a drug and/or health care product for decreasing cytokines contents of immune interleukin-6 and interleukin-2.

Compared with the prior art, the present disclosure has the following beneficial technical effects.

The formula for treating the menopause, the menopausal mental depression and the atypical depressive disorder disclosed in the present disclosure is composed of the sea buckthorn pulp oil and the total panax notoginseng saponins from stem and leave serving as the active ingredients according to a certain ratio (70%-97.5% of the sea buckthorn pulp oil and 2.5%-30% (g/g) of the total panax notoginseng saponins from stem and leave). Through experimental verifications, compared with a rat model group of a mental depression model of as well as positive drugs of non-tricyclic western medicine of paroxetine and Xiaoyao pills, the natural pharmaceutical composition has obvious specific effects as follows. The natural pharmaceutical composition can effectively 1) regulate a ratio of contents of signal molecules cyclic adenosine monophosphate (CAMP) to cyclic guanosine monophosphate (CGMP); 2) regulate a ratio of contents of a sex hormone dihydrotestosterone to a female hormone; 3) regulate a ratio of a sex hormone follicle-stimulating hormone (FSH) to oxytocin (OT); 4) regulate a ratio of a neurotransmitter noradrenaline NA to dopamine and serotonin; and 5) decrease contents of the immune cytokines of interleukin-6 and interleukin-2 in serum of rats of the mental depression model. The present disclosure is a natural drug formula with homology of medicine and food, is reasonable in compatibility and safe in dose, does not have any side effect on a human body, overcomes defects of clinically common monoamine oxidase inhibitors and non-tricyclic drugs such as the paroxetine, and has fast remarkable effects and obvious curative effects. Serum marker indexes of the present disclosure are obvious and better than those of similar traditional Chinese medicines and some synthetic chemicals for treating the menopause, the menopausal mental depression and the atypical depressive disorder in a market. Problems that the existing drugs have side effects and poor curative effects in treating the menopause, the menopausal mental depression and the atypical depressive disorder can be effectively solved.

### DETAILED DESCRIPTION OF THE DISCLOSURE

The present disclosure is further described in detail below in combination with specific embodiments. The description is an explanation of, but not a limitation to, the present disclosure.

1. Preparation Method of Formula Ingredients, Formula Ratio Range and Daily Dose-Effect Ratio

A natural pharmaceutical composition for treating mental depression and atypical depressive disorder disclosed in the present disclosure is composed of the following components in percentage by mass: 70%-97.5% of sea buckthorn pulp oil and 2.5%-30% (g/g) of total panax notoginseng saponins from stem and leave.

Three preparation methods of active ingredients in the present disclosure can be adopted as follows.

1) Preparation of the sea buckthorn pulp oil comprises the following common methods:

a. centrifugal separation by physical pressing: pressing (de-seeding) fresh mature sea buckthorn fruits (without seeds) to obtain sea buckthorn juice and sea buckthorn fruit pulp residues, adding warm water of 80° C. into the fruit pulp residues for 1.5 h, and performing centrifugal separation for different times, thereby obtaining the sea buckthorn pulp oil with an oil extraction rate of 1.6%-2.2% (g/g);

b. CO₂ supercritical extraction: performing constant-temperature drying on the fresh mature sea buckthorn fruits (without seeds) to obtain dried sea buckthorn fruit pulp residues, wherein extraction conditions of industrial production are as follows: an extraction pressure of 35 MPa, an extraction temperature of 45° C., extraction time of 1.5 h, and a separation temperature of 45° C., and the oil extraction rate can reach 5.0%-5.6% (g/g); adding water according to a ratio of 1:1 to hydrate and remove impurities, adding 3%-5% of argil to discolor, and performing centrifugal dehydration, thereby obtaining 87% (g/g) of purified and refined sea buckthorn pulp oil; and

c. extraction with an n-hexane solvent: moderately soaking dried sea buckthorn fruits, and extruding the fruits (removing the seeds) by a roller to obtain the fruit pulp residues; drying at constant temperature of 45-55° C., enabling the sea buckthorn fruit pulp residues to reach a granularity of 40 meshes, extracting with the n-hexane solvent for 50 minutes according to a ratio 1:8 of the material to the solvent at an extraction temperature of 60° C. to obtain sea buckthorn fruit oil with an extraction rate of 4.8-5.3% (g/g); and purifying and refining, thereby obtaining 86% (g/g) of purified and refined sea buckthorn pulp oil. The sea buckthorn pulp oil contains myristic acid C14:0, palmitic acid C16:0, palmitoleic acid C16:1, heptadecanoic acid C17:0, oleic acid C18:1, linoleic acid C18:2, linolenic acid C18:3, arachidic acid C20:0, β-sitosterol, stigmasterol, seed phytosterol, ergosterol, diterpene ester, triterpene ester, sterol ester, β-carotene, xanthophylls, quercetin kaempferol, isorhamnetin, vitamin E, vitamin K and other components.

2) Preparation of the Total Panax Notoginseng Saponins from Stem and Leave

Traditional alcohol extraction of the total panax notoginseng saponins from stem and leave at an effective part of leaves of panax notoginseng: extracting 40-mesh panax notoginseng powder with 80% of ethanol solution according to a material ratio of 1:7, and placing in a container for performing reflux extraction, performing boiling reflux extraction twice, extracting for 1.5 hours each time, merging filtrate to reach a rate of 9.6% (g/g), concentrating through HPD-400 macroporous resin, eluting with 80% of ethanol, and concentrating and drying the eluent, thereby obtaining the purified total panax notoginseng saponins from stem and leave product. The total panax notoginseng saponins from stem and leave contains panaxoside Rb1, panaxoside Rb3, trace Dama-20(22)-ene-β, 12β, 26-triol 20(R)-dammarane-3β, and the like.

Compared with a water extraction method adopted in Chinese Pharmacopoeia of 2015 version, the extraction method of 80% ethanol solution has slightly higher yield of the total panax notoginseng saponins from stem and leave than that of the water extraction method, while with respect to economic cost, the water extraction method is economic and cheap.

2. Ratio of a Compound Pharmaceutical Composition as Well as Medication Method and Treatment Cycle (Formula Cases)

1) Formula Cases

### Embodiment 1

A natural pharmaceutical composition for treating menopause and/or menopausal mental depression and atypical depressive disorder is a drug prepared from the following active ingredients in percentage by mass: 96.7% of sea buckthorn pulp oil and 3.3% of total panax notoginseng saponins from stem and leave.

### Embodiment 2

A natural pharmaceutical composition for treating menopause and/or menopausal mental depression and atypical depressive disorder is a drug prepared from the following active ingredients in percentage by mass: 84.0% of sea buckthorn pulp oil and 16.0% of total panax notoginseng saponins from stem and leave.

### Embodiment 3

A natural pharmaceutical composition for treating menopause and/or menopausal mental depression and atypical depressive disorder is a drug prepared from the following active ingredients in percentage by mass: 90.0% of sea buckthorn pulp oil and 10.0% of total panax notoginseng saponins from stem and leave.

### Embodiment 4

A natural pharmaceutical composition for treating menopause and/or menopausal mental depression and atypical depressive disorder is a drug prepared from the following active ingredients in percentage by mass: 92.7% of sea buckthorn pulp oil and 7.3% of total panax notoginseng saponins from stem and leave.

### Embodiment 5

A natural pharmaceutical composition for treating menopause and/or menopausal mental depression and atypical depressive disorder is a drug prepared from the following active ingredients in percentage by mass: 70.0% of sea buckthorn pulp oil and 9.0% of total panax notoginseng saponins from stem and leave.

### Embodiment 6

A natural pharmaceutical composition for treating menopause and/or menopausal mental depression and atypical depressive disorder is a drug prepared from the following active ingredients in percentage by mass: 92.5% of sea buckthorn pulp oil and 7.5% of total panax notoginseng saponins from stem and leave.

2) Ratio, Daily Dose and Medication Cycle of the Compound Pharmaceutical Composition

A combined ratio of drugs of the sea buckthorn pulp oil to total panax notoginseng saponins from stem and leave is (weight/weight) % (g/g).

A ratio of the sea buckthorn pulp oil to the total panax notoginseng saponins from stem and leave is in a range of (70.0%-97.5%):(2.5%-30%).

A daily dose range of a prepared drug composition composed of the sea buckthorn pulp oil and the total panax notoginseng saponins from stem and leave for animal rats is (0.5-3.0)g: (0.05-0.15) g/kg.

A dose-effect daily dose of the rat of the product is: a range of 1.0 g:0.1 g/kg or 0.6 g:0.06 g/kg of the sea buckthorn pulp oil to the total panax notoginseng saponins from stem and leave is an optimal ratio range for the remarkable effects.

A human clinical equivalent formula ratio is as follows.

a ratio of the sea buckthorn pulp oil to the total panax notoginseng saponins from stem and leave is in a range of (70%-97.5%):(2.5%-30%).

A human (g/60 g) daily dose range is: a ratio of the sea buckthorn pulp oil to the total panax notoginseng saponins from stem and leave is (5 g-30 g):(0.5 g-1.5 g)/60 kg.

A human daily dose of the product is: a range of (10.0 g:1.0 g)/60 kg or (6 g:0.6 g)/60 kg of the sea buckthorn pulp oil to the total panax notoginseng saponins from stem and leave is an optimal ratio range for the remarkable effects, wherein interaction strength of the (10.0 g:1.0 g)/60 kg is higher than that of the (6 g:0.6 g)/60 kg.

A medication cycle of the present compound drug is 3 months, and neuroendocrine, hormone, cyclic nucleotide molecule and behavioral disorders can be regulated to be normal.

3. Characteristics of Serum Marker Indexes (Neuroendocrine, Hormone and Cyclic Nucleotide Molecules) of the Rat of the Compound Drug Model

1) comparing the compound drug group with a menopause group and a menopausal depression model group, a content ratio of a second messenger cyclic adenosine monophosphate (CAMP) to cyclic guanosine monophosphate (CGMP) of serum protein receptors of rats suffering from the menopause and menopausal mental depression is increased from 1.4:1 (a disorder value) to 2.5:1 (close to a normal and healthy value);

2) comparing the compound drug group with two model groups, a ratio of dihydrotestosterone to estradiol of the rats suffering from the menopause and the menopausal mental depression is decreased from 2.0-1.25:1 (a disorder value) to 1.05-1.0:1 (a normal and healthy value);

3) comparing the compound drug group with the two model groups, content of the follicle-stimulating hormone is decreased, a ratio of the oxytocin (a love hormone) OT is increased, and a ratio of FSH 2.0-1.44 (a disorder value):(OT)1.0 is decreased to 1.05-1.0 (FSH): 1(OT) (a normal and healthy value);

4) comparing the compound drug group with the two model groups, a neurotransmitter disorder state ratio of norepinephrine (NA) to dopamine (DA) in the rats suffering from the menopause and the menopausal mental depression is decreased from 7.0-6.0:1 (a disorder value) to 3.0-2.0:1 (restored to a normal and healthy value); and

5) comparing the compound drug group with the two model groups, a ratio of the content of the disorder state dihydrotestosterone DHT to female hormone in the rats suffering from the menopause and the menopausal mental depression is decreased from 2:1 (a disorder value) to 1.04:1 (a normal and healthy value).

Through the increase or decrease of the ratio of the CAMP to CGMP, the ratio of the sex hormones FSH to OT, the ratio of the DHT to E2, the ratio of the neurotransmitters NA to DA and the content ratio in the above characteristics, regulation from the disorder state to the healthy state is an advantage characteristic of the compound drug in treating the menopause and the menopausal mental depression.

According to the characteristics of the compound drug group for regulating the serum marker indexes (neuroendocrine, hormone and cyclic nucleotide signal molecules) of the menopause and the menopausal mental depression, behavior abnormalities are caused in the menopause group and the menopausal depression model group due to severe disorders of internal secretion influencing factors such as the neurotransmitters, the hormones, the cyclic nucleotide signal molecules and the like. The compound drug composed of the drugs of the sea buckthorn pulp oil and total panax notoginseng saponins from stem and leave in the present disclosure can effectively regulate mutual content ratio relationships among internal secretion marker signal molecules and influencing factors such as the neurotransmitters, the hormones, the cyclic nucleotide signal molecules and the like in the serum of the menopause and mental depression model rats, and correct and treat the behavior abnormalities of the menopause and the mental depression.

4. Behavior Examination Results after Medication

1) Behavioral Contrast Results of Each Group within 3 Cycles Before and after Medication

Open-field test results (Table 1) show that: horizontal scores of a compound high-dose group, a Xiaoyao pill group and a paroxetine group after medication are obviously higher than scores before medication (P<0.01), horizontal scores of a compound medium-dose group and a compound low-dose group after medication are higher than scores before medication, and statistical significance is achieved (P<0.05); and horizontal scores of the menopause group, the menopausal depression group and a sham-operated group before and after medication do not have any obvious difference.

A vertical score of the compound medium-dose group after medication is higher than a score before medication, and statistical significance is achieved (P<0.05); and vertical scores of the rest medication groups, the pre-menopause group, the menopausal depression group and the sham-operated group before and after medication do not have any obvious difference. With respect to fecal weight, any significant difference does not exist in various medication groups, the pre-menopause group, the menopausal depression group and the sham-operated group before and after medication.

TABLE 1
Statistical results of open-field tests of rats
in various groups before and after medication

| | | | After | |
|---|---|---|---|---|
| | | After medication | medication | After |
| | | minus before | minus before | medication |
| | | medication | medication | minus Before |
| | | (horizontal | (vertical | medication |
| Groups | Cases | scores) | scores) | (Fecal weight) |
| Compound | 8 | 31.29 ± 14.35** | 2.43 ± 7.12 | −1.01 ± 0.65** |
| high-dose | | | | |
| group | | | | |
| Compound | 8 | 41.25 ± 39.10* | 4.12 ± 4.22* | −0.19 ± 0.71 |
| medium- | | | | |
| dose | | | | |
| group | | | | |
| Compound | 8 | 34.38 ± 37.10* | 4.00 ± 8.38 | −0.99 ± 1.26 |
| low-dose | | | | |
| group | | | | |
| Xiaoyao pill | 8 | 51.88 ± 12.05** | 7.75 ± 17.86 | −0.65 ± 0.73* |
| Paroxetine | 8 | 41.14 ± 28.54** | −3.00 ± 10.49 | −0.94 ± 0.71* |
| Menopausal | 8 | 1.38 ± 26.44 | −8.88 ± 10.56 | −4.62 ± 0.69 |
| depression | | | | |
| Menopause | 8 | 13.08 ± 23.87 | −2.57 ± 5.53 | −0.23 ± 0.39 |
| Sham- | 8 | 21.50 ± 29.88 | 1.33 ± 7.89 | −1.20 ± 0.88* |
| operated | | | | |
| group | | | | |

Notes:
*P < 0.05,
**P < 0.01

Experimental results (Table 2) show that: water drinking latent periods of a compound high-dose group, a compound medium-dose group, a compound low-dose group, a Xiaoyao pill group and a paroxetine group after medication are obviously higher than scores before medication (P<0.01), water drinking latent periods of the menopause group and the sham-operated group after medication are higher than scores before medication, and statistical significance is achieved (P<0.05); and water drinking latent periods of the menopausal depression group before and after medication do not have any obvious difference.

Water drinking time scores of the compound medium-dose group, the compound low-dose group, the Xiaoyao pill group and the paroxetine group after medication are obviously higher than scores before medication (P<0.01), water drinking time scores of a hybrid high-dose group and the sham-operated group after medication are higher than scores before medication, and statistical significance is achieved (P<0.05); and water drinking time of the menopausal depression group before and after medication does not have any obvious difference.

TABLE 2
Statistical results of open-field water drinking tests of rats
in various groups before and after medication

| | | After medication | After |
|---|---|---|---|
| | | minus Medication | medication minus |
| | | (Water drinking | Before medication |
| Groups | Cases | latent period) | (Water drinking time) |
| Compound | 8 | 51.712 ± 25.77** | 34.00 ± 27.10* |
| high-dose | | | |
| group | | | |
| Compound | 8 | 79.38 ± 40.49** | 103.62 ± 76.91** |
| medium-dose | | | |
| group | | | |
| Compound | 8 | 50.62 ± 38.72** | 63.25 ± 44.92** |
| low-dose group | | | |
| Xiaoyao pill | 8 | 121.86 ± 78.59** | 77.75 ± 28.52** |
| Paroxetine | 8 | 70.43 ± 35.65** | 112.14 ± 50.83** |
| Menopausal | 8 | 7.25 ± 16.56 | 20.75 ± 18.39* |
| depression | | | |
| Menopause | 8 | −125.43 ± 148.73* | 19.43 ± 56.00 |
| Sham-operated | 8 | −108.51 ± 96.50* | 78.33 ± 61.38* |
| group | | | |

Notes:
*P < 0.05,
**P < 0.01

As shown in Table 3 (continued), frequencies of approaching water bottles in the compound medium-dose group, the compound low-dose group and the Xiaoyao pill group after medication are obviously higher than scores before medication (P<0.01), frequencies of approaching water bottles in the compound high-dose group and the paroxetine group after medication are higher than scores before medication, and statistical significance is achieved (P<0.05); and the frequencies of approaching water bottles in the menopause group, the sham-operated group and the menopausal depression group before and after medication do not have any obvious difference.

Standing frequencies of the compound medium-dose group, the Xiaoyao pill group and the paroxetine group after medication are obviously higher than scores before medication (P<0.01), the standing frequencies of the compound low-dose group after medication are higher than scores before medication, and statistical significance is achieved (P<0.05); and the standing frequencies of the compound high-dose group, the menopause group, the sham-operated group and the menopausal depression group before and after medication does not have any obvious difference.

TABLE 3
(Continued) Statistical results of open-field water drinking tests
of rats in various groups before and after medication

| | | | After medication |
|---|---|---|---|
| | | After medication minus | minus Before |
| | | Before medication | medication |
| | | (Frequencies of | (Standing |
| Groups | Cases | approaching water bottles) | frequency) |
| Compound | 8 | 3.8622 ± 3.6303* | −0.8602 ± 3.8525 |
| high-dose | | | |
| group | | | |
| Compound | 8 | 11.2502 ± 6.2303** | 14.1335 ± 5.2843** |
| medium-dose | | | |
| group | | | |
| Compound | 8 | 3.8806 ± 2.3223** | 5.8802 ± 5.3853* |
| low-dose group | | | |
| Xiaoyao pill | 8 | 4.5042 ± 2.4523** | 10.5057 ± 7.3335** |
| Paroxetine | 8 | 6.5742 ± 4.0443* | 7.5728 ± 4.5413** |
| Menopausal | 8 | −0.6225 ± 2.2638 | 2.7550 ± 7.4017 |
| depression | | | |
| Menopause | 8 | −1.5734 ± 3.5558 | −4.4314 ± 5.2235 |
| Sham-operated | 8 | −0.8330 ± 3.6612 | −2.3315 ± 6.27771 |
| group | | | |

Notes:
*P < 0.05,
**P < 0.01

2) Behavioral Comparison Results of Each Group and the Menopausal Depression Group after Medication

a. Determination Results of Open-Field Test

Experimental results (Table 4) show that: compared with the sham-operated group, the horizontal scores of the model groups are obviously decreased (P<0.05), the horizontal scores in the low-dose group, the paroxetine group and the menopause group are obviously higher than those in the model groups (P<0.01), the horizontal scores in the compound high-dose group, the compound medium-dose group and the Xiaoyao pill group are higher than those in the model groups, and statistical significance is achieved (P<0.05).

Compared with the sham-operated group, the vertical scores of the model groups have the statistical significance (P<0.05), wherein the vertical score in the menopause group is obviously higher than those in the model groups (P<0.01), the vertical scores in the compound high-dose group, the compound medium-dose group, the compound low-dose group and the Xiaoyao pill group are higher than those in the model groups, and statistical significance is achieved (P<0.05); the vertical score in the paroxetine group does not have any obvious difference from the vertical score in the model group; and with respect to the fecal weight, any significant difference does not exist in various medication groups and model groups.

TABLE 4
Changes of horizontal scores, vertical scores and fecal weight
of open-field tests of rats in various groups

| Groups | Cases | Horizontal scores | Vertical scores | Fecal weight |
|---|---|---|---|---|
| Compound | 8 | 53.86 ± 11.14* | 14.57 ± 4.54* | 0.10 ± 0.26 |
| high-dose | | | | |
| group | | | | |
| Compound | 8 | 68.00 ± 33.72* | 13.87 ± 4.39* | 0.26 ± 0.39 |
| medium- | | | | |
| dose group | | | | |
| Compound | 8 | 73.87 ± 22.24** | 17.12 ± 6.94* | 0.00 ± 0.00 |
| low-dose | | | | |
| group | | | | |
| Xiaoyao pill | 8 | 71.12 ± 14.08* | 18.62 ± 8.28* | 0.16 ± 0.31 |
| Paroxetine | 8 | 78.29 ± 28.18** | 9.57 ± 4.68 | 0.14 ± 0.38 |
| Menopausal | 8 | 34.62 ± 26.27- | 8.50 ± 10.86- | 0.00 ± 0.00 |
| depression | | | | |
| Menopause | 8 | 85.43 ± 30.15** | 28.14 ± 12.28** | 0.00 ± 0.00 |
| Sham- | 8 | 71.80 ± 21.52* | 19.80 ± 3.19* | 0.00 ± 0.00 |
| operated | | | | |
| group | | | | |

Notes:
compared with the menopause model group, *P < 0.05, **P < 0.01

b. Determination Results of Open-Field Water Drinking Test

Experimental results (Table 5) show that: compared with the sham-operated group, the water drinking latent periods in the model groups are obviously decreased (P<0.01), the water drinking latent periods in the compound medium-dose group, the Xiaoyao pill group, the paroxetine group and the menopause group are obviously higher than those in the model groups (P<0.01), the water drinking latent period in the low-dose group is higher than that in the model group, statistical significance is achieved (P<0.05), and the water drinking latent period in the high-dose group does not have any significant difference from the model group.

Compared with the sham-operated group, the water drinking time in the model group is obviously decreased (P<0.01), wherein the water drinking time in the Xiaoyao pill group and the paroxetine group is obviously higher than those in the model groups (P<0.01), the water drinking time in the compound medium-dose group and the compound low-dose group is higher than those in the model groups, the statistical significance is achieved (P<0.05), and the water drinking time in the high-dose group does not have any significant difference from the model groups.

TABLE 5
Changes of water drinking latent periods, water drinking time,
frequencies of approaching water bottles and standing frequencies
of open-field water drinking tests of rats in various groups

| | | | Water |
|---|---|---|---|
| Groups | Cases | Water drinking latent period | drinking time |
| Compound | 8 | 54.00 ± 26.11 | 34.00 ± 27.10 |
| high-dose | | | |
| group | | | |
| Compound | 8 | 82.25 ± 40.56** | 103.62 ± 76.91* |
| medium-dose | | | |
| group | | | |
| Compound | 8 | 63.25 ± 35.60* | 63.50 ± 44.63* |
| low-dose group | | | |
| Xiaoyao pill | 8 | 123.12 ± 80.29** | 77.75 ± 28.51** |
| Paroxetine | 8 | 88.86 ± 20.05** | 112.14 ± 50.83** |
| Menopausal | 8 | 27.62 ± 20.58- | 20.75 ± 18.39- |
| depression | | | |
| Menopause | 8 | 124.00 ± 57.88** | 59.57 ± 46.75* |
| Sham-operated | 8 | 198.17 ± 68.49** | 136.17 ± 61.62** |
| group | | | |

Notes:
compared with the menopause model group, *P < 0.05, **P < 0.01

As shown in Table 6 (continued): compared with the sham-operated group, the frequencies of approaching water bottles in the model groups are obviously decreased (P<0.01), the frequencies of approaching water bottles in the compound medium-dose group, the paroxetine group and the menopause group are obviously higher than those in the model groups (P<0.01), and the frequencies of approaching water bottles in the compound high-dose group, the compound low-dose group and the Xiaoyao pill group do not have any significant difference from the model groups.

Compared with the sham-operated group, the standing frequency in the model group is decreased, and the statistical significance is achieved (P<0.05); the standing frequency in the menopause group is higher than that in the model group (P<0.01), the standing frequency in the compound medium-dose group is higher than that in the model group, and the statistical significance is achieved (P<0.05); and the standing frequencies in the compound high-dose group, the compound low-dose group, the Xiaoyao pill group and the paroxetine group do not have any significant difference from those in the model groups.

TABLE 6
(Continued)

| | | Number of times | |
|---|---|---|---|
| | | approaching | Number of |
| Groups | Cases | to water bottles | times standing |
| Compound | 8 | 4.86 ± 3.58 | 2.86 ± 1.86 |
| high-dose group | | | |
| Compound | 8 | 12.12 ± 6.36** | 16.00 ± 5.42* |
| medium-dose | | | |
| group | | | |
| Compound | 8 | 5.25 ± 2.25 | 8.75 ± 3.95 |
| low-dose group | | | |
| Xiaoyao pill | 8 | 5.50 ± 2.67 | 13.62 ± 6.52 |
| Paroxetine | 8 | 10.43 ± 5.09** | 11.28 ± 4.23 |
| Menopausal | 8 | 3.62 ± 2.26- | 7.87 ± 6.40- |
| depression | | | |
| Menopause | 8 | 8.57 ± 2.93** | 19.57 ± 3.36** |
| Sham-operated | 8 | 11.67 ± 4.13** | 15.67 ± 3.78* |
| group | | | |

Notes:
compared with the menopause model group, *P < 0.05, **P < 0.01

5. Regulation Changes of Marker Indexes Such as Neuroendocrine, Hormone and Cyclic Nucleotide Signal Molecules in Serum of Rats

1) Specimen Collection and Treatment Methods

drenching animals in sequence to put to death within 3 cycles, enabling the animals to have empty stomachs for 12 hours before putting to death, and performing intraperitoneal anesthesia with 3% of pentobarbital sodium at a dose of 0.1 ml/100 g; and taking 8 ml of whole blood in a centrifuge tube, centrifuging at a rate of 4000 r/min for 15 min, separating the serum after centrifuging, labeling, and separately filling and preserving in a refrigerator of −20° C.;

2) ELISA Determination Method is as Follows:

Detection is performed according to description of an ELISA kit. Before use, all reagents are fully and uniformly mixed. Lots of bubbles should not be produced in liquid, so as to avoid generating a sample adding error due to addition of lots of bubbles during sample adding. The quantity of needed plates is determined according to a quantity of to-be-detected samples and the quantity of standard substances. Each standard substance and each blank hole need to be provided with duplicate holes. However, since many samples need to be determined, single holes should be made according to actual conditions.

Step 1: respectively setting blank holes (any sample or enzyme-labeled reagent is not added into each blank control hole, and operations in the rest various steps are the same), standard holes and to-be-detected sample holes; adding 50 ul of the diluted standard substance into the reaction holes on an enzyme-labeled coated plate, adding 50 ul of the to-be-detected sample into the to-be-detected sample holes; adding the samples to the bottom of the coated holes during sample adding as much as possible, enabling the samples to be not contacted with holes as much as possible, and slightly shaking to be uniform; and covering a micro-plate sealer, and incubating at 37° C. for 30 min.

Step 2: diluting a scrubbing solution with distilled water by 30 times for later use;

carefully uncovering the micro-plate sealer, throwing out liquid in the holes, enabling each hole to be filled with the scrubbing solution, oscillating for 30 seconds, throwing out the scrubbing solution, and patting dry with absorbent paper; and repeating the operation for 5 times.

Step 3: adding 50 ul of the enzyme-labeled reagent into each hole except the blank holes, slightly shaking and mixing uniformly, and incubating at 37° C. for 30 min;

carefully uncovering the micro-plate sealer, throwing out the liquid in the holes, enabling each hole to be filled with the scrubbing solution, oscillating for 30 seconds, throwing out the scrubbing solution, and patting dry with absorbent paper; and repeating the operation for 5 times.

Step 4, respectively adding 50 ul of color developing agents A and B into each hole, slightly shaking and mixing uniformly, and incubating at 37° C. to be kept in a dark place and developed for 10 min.

Step 5: taking out the ELISA plate, rapidly adding 50 ul of stop buffer, and immediately determining the results after the stop buffer is added; resetting at the blank holes, and determining a value OD at each hole at a wavelength of 450 nm; and

Step 6: calculating a regression equation of a standard curve by using the concentration of the standard substance and the value OD, and drawing a standard curve according to the concentration of the standard substance and D(λ); obtaining a numerical value in each hole according to the standard curve; and substituting the value OD of the sample into the equation, thereby calculating the sample concentration.

A basic principle of enzyme-labeled immunoassay is as follows: an antibody connected onto a solid-phase carrier and an enzyme-labeled antibody are respectively bound to two antigenic determinants on a detected antigen molecule in the sample to form a solid-phase antibody-antigen-enzyme-labeled antibody immune complex. Since the quantity of the solid-phase antibody and the enzyme-labeled antibody in a reaction system is excessive relative to a to-be-detected antigen, a formative amount of the complex is in direct proportion to content of the to-be-detected antigen (in a detectable range of the method). An amount of a colored matter (the value OD) produced by enabling the enzyme in the complex to act on the added substrate is determined, thereby determining the content of the to-be-detected antigen. The method has advantages of high stability, long preservation period, easy and convenient operation, high specificity and zero radionuclide contamination.

3) Determination Results of Serum Biochemical Indicators

a. Influence of Each Test Drug on Adrenocorticotrophic Hormone

Experimental results (Table 7) show that: compared with the menopausal depression group, the compound high-dose group, the compound medium-dose group and (a normal group) the sham-operated group have obviously decreased content of adrenocorticotrophic hormone in serum (P<0.05); compared with the menopausal depression group, other medication groups have a decreasing tendency of the content of the adrenocorticotrophic hormone, but no statistical significance is achieved.

TABLE 7
Influence of the drug on content of adrenocorticotrophic hormone of rats

| Groups | Cases | ACTH (ng/L) |
|---|---|---|
| Compound high-dose group | 6 | 14.2552 ± 2.2376* |
| Compound medium-dose | 6 | 15.0231 ± 3.1218* |
| group | | |
| Compound low-dose group | 6 | 15.2700 ± 3.0771 |
| Xiaoyao pill | 6 | 15.0353 ± 1.4447 |
| Paroxetine | 6 | 16.3682 ± 1.8767 |
| Sham-operated group | 6 | 12.7313 ± 0.9822* |
| Menopause | 6 | 17.7609 ± 2.8420 |
| Menopausal depression | 6 | 19.2542 ± 3.0815 |

Notes:
compared with the menopausal depression group, *P < 0.05, **P < 0.01

b. Influence of Each Test Drug on Monoamine Neurotransmitter

Experimental results (Table 8) show that: compared with the menopausal depression group, the compound high-dose group, the compound medium-dose group and the sham-operated group have obviously increased content of noradrenaline in serum (P<0.01); compared with the menopausal depression group, the compound low-dose group and the paroxetine group have increased content of the noradrenaline in the serum, and the statistical significance is achieved (P<0.05); and compared with the menopausal depression group, the Xiaoyao pill medication group and the menopause group have an increasing tendency of the content of the noradrenaline in the serum, but no statistical significance is achieved.

Compared with the menopausal depression group, the compound high-dose group has obviously increased content of 5-hydroxytryptamine in the serum (P<0.01); compared with the menopausal depression group, the Xiaoyao pill group and the sham-operated group have increased content of the 5-hydroxytryptamine in the serum, and the statistical significance is achieved (P<0.05); and compared with the menopausal depression group, no statistical significance is achieved in the rest of various medication groups.

Compared with the menopausal depression group, the compound high-dose group, the compound low-dose group, the compound low-dose group and the sham-operated group have obviously increased content of dopamine in the serum (P<0.05); and compared with the menopausal depression group, no statistical significance is achieved in the rest of the various medication groups.

TABLE 8
Influences of the drug on the content of noradrenaline, 5-hydroxytryptamine and
dopamine in serum of rats

| Groups | Cases | NA (ng/L) | 5-HT (ng/L) | DA (pg/L) |
|---|---|---|---|---|
| Compound | 6 | 36.1173 ± 8.1370** | 50.4782 ± 4.6096** | 8.9122 ± 0.6768* |
| high-dose | | | | |
| group | | | | |
| Compound | 6 | 33.7038 ± 8.8206** | 42.4312 ± 5.5918 | 8.9526 ± 1.2471* |
| medium-dose | | | | |
| group | | | | |
| Compound | 6 | 30.7078 ± 8.2948* | 44.6567 ± 10.4979 | 8.4437 ± 0.794* |
| low-dose group | | | | |
| Xiaoyao pill | 6 | 23.2608 ± 3.0599 | 51.9585 ± 8.5655* | 7.0219 ± 0.5231 |
| Paroxetine | 6 | 32.5359 ± 5.6128* | 42.9048 ± 6.2922 | 7.6595 ± 1.3337 |
| Sham-operated | 6 | 42.6441 ± 2.8624** | 45.8641 ± 4.9117* | 9.6148 ± 0.7971* |
| group | | | | |
| Menopause | 6 | 25.2227 ± 4.8524 | 39.1468 ± 8.9903 | 6.6355 ± 0.9681 |
| Menopausal | 6 | 19.6333 ± 2.8633 | 36.3836 ± 5.276 | 6.5963 ± 1.5534* |
| depression | | | | |

Notes:
compared with the menopausal depression group, *P < 0.05, **P < 0.01

c. Influence of Each Test Drug on cAMP/cGM

Results in Table 9 show that: compared with the menopausal depression group, the compound high-dose group, the compound medium-dose group, the Xiaoyao pill group and the sham-operated group have obviously increased content of cAMP in serum (P<0.05); compared with the menopausal depression group, the compound low-dose group and the sham-operated group have obviously decreased content of cGMP in the serum (P<0.01); compared with the menopausal depression group, the compound medium-dose group and the paroxetine have decreased content of the cGMP in the serum, and the statistical significance is achieved (P<0.05); compared with the menopausal depression group, the compound high-dose group, the compound medium-dose group, the compound low-dose group and the sham-operated group have obviously increased content of cAMP/cGMP in the serum (P<0.01); and compared with the menopausal depression group, the Xiaoyao pill group has increased content of the cAMP/cGMP in the serum, and the statistical significance is achieved (P<0.05).

TABLE 9
Influences of the drug on the content of cAMP, cGMP and cAMP/cGMP ratio in
serum of rats

| Groups | Cases | cAMP (U/L) | cGMP (U/L) | cAMP/cGMP (U/nmol) |
|---|---|---|---|---|
| Compound | 6 | 5.3920 ± 0.7205* | 2.0177 ± 0.3595 | 2.7430 ± 0.5961** |
| high-dose group | | | | |
| Compound | 6 | 4.6861 ± 0.7344* | 1.9347 ± 0.4104* | 2.4575 ± 0.3139** |
| medium-dose | | | | |
| group | | | | |
| Compound | 6 | 4.5387 ± 0.7699* | 1.6993 ± 0.2208** | 2.6821 ± 0.4192** |
| low-dose group | | | | |
| Xiaoyao pill | 6 | 4.7805 ± 0.6356* | 2.0634 ± 0.2623 | 2.3746 ± 0.6173* |
| Paroxetine | 6 | 2.7761 ± 0.3726 | 1.8333 ± 0.4686* | 1.5777 ± 0.3723 |
| Sham-operated | 6 | 4.7931 ± 1.0499* | 1.6763 ± 0.1614** | 2.9074 ± 0.7892** |
| group | | | | |
| Menopause | 6 | 3.3888 ± 0.6339 | 2.3945 ± 0.8105 | 1.6645 ± 1.0251 |
| Menopausal | 6 | 3.2939 ± 0.6092 | 2.7603 ± 0.5602 | 1.2143 ± 0.2502 |
| depression | | | | |

Notes:
compared with the menopausal depression group, *P < 0.05, **P < 0.01

Results in Table 10 show that: compared with the menopausal depression group, the compound low-dose group has obviously decreased content of follicle-stimulating hormone (FSH) in serum (P<0.05); compared with the menopausal depression group, no statistical significance is achieved in the rest of the various groups; compared with the menopausal depression group, the compound medium-dose group has obviously increased content of oxytocin (OT) in the serum (P<0.05); and in the compound high/medium/low-dose groups of the test drug, a ratio 2:1 (a model-group disorder state) of the FSH to the OH in the serum of the rats suffering from the menopausal depression may be regulated to be about 1:1 (normal in the compound drug group and the normal group and sham-operated group) after drug medication.

TABLE 10
Influences of the drug on the content of oxytocin and
follicle-stimulating hormone in serum of rats

| Groups | Cases | OT (ng/L) | FSH (IU/L) |
|---|---|---|---|
| Compound | 6 | 4.1573 ± 1.0252 | 4.3607 ± 1.3979 |
| high-dose group | | | |
| Compound | 6 | 5.2112 ± 0.883* | 3.6322 ± 0.9354 |
| medium-dose | | | |
| group | | | |
| Compound | 6 | 3.7536 ± 0.7703 | 3.365 ± 0.9938* |
| low-dose group | | | |
| Xiaoyao pill | 6 | 4.7159 ± 0.8251 | 4.6173 ± 0.3324 |
| Paroxetine | 6 | 4.1889 ± 0.8811 | 3.7068 ± 0.6242 |
| Sham-operated | 6 | 4.4236 ± 0.3904 | 4.4381 ± 3.2524 |
| group | | | |
| Menopause | 6 | 4.0877 ± 0.8027 | 5.6247 ± 4.0935 |
| Menopausal | 6 | 3.8041 ± 0.8939 | 7.7284 ± 4.7723 |
| depression | | | |

Notes:
compared with the menopausal depression group, *P < 0.05, **P < 0.01

Results in Table 11 show that: compared with the menopausal depression group, the compound medium-dose group has obviously increased content of dihydrotestosterone (DHT) in serum (P<0.05); compared with the menopausal depression group, the rest of the medication groups have an increasing tendency of the content of the DHT in serum, but no statistical significance is achieved; and compared with the menopause group and the menopausal depression group, a ratio relationship of the DHT to estrogen shows that a ratio of the content of the DHT to the content of the E2 in the serum of rats suffering from the menopause and menopausal depression is regulated from 2:1 (a disorder state) to about 1.1:1 (a normal state).

TABLE 11
Influences of the drug on the content of
dihydrotestosterone and estradiol in serum of rats

| Groups | Cases | DHT (nmol/L) | E2 (ng/L) |
|---|---|---|---|
| Compound | 6 | 10.4036 ± 1.2949 | 14.0828 ± 2.3138 |
| high-dose group | | | |
| Compound | 6 | 12.5223 ± 2.1372* | 14.3164 ± 1.7679 |
| medium-dose | | | |
| group | | | |
| Compound | 6 | 10.6069 ± 1.3551 | 12.4852 ± 1.4197 |
| low-dose group | | | |
| Xiaoyao pill | 6 | 9.4591 ± 2.3601 | 14.5805 ± 3.8365 |
| Paroxetine | 6 | 10.5073 ± 1.9504 | 10.0566 ± 0.7140 |
| Sham-operated | 6 | 13.3734 ± 4.3305* | 14.9538 ± 1.1187 |
| group | | | |
| Menopause | 6 | 7.5176 ± 2.5299 | 10.9474 ± 2.2553 |
| Menopausal | 6 | 7.8304 ± 1.5839 | 11.4818 ± 2.2599 |
| depression | | | |

Notes:
compared with the menopausal depression group, *P < 0.05, **P < 0.01

Results in Table 12 show that: compared with the menopausal depression group, the compound high-dose group, the compound medium-dose group, the compound low-dose group, the paroxetine group and the sham-operated group have obviously decreased content of interleukin-6 in serum (P<0.01); compared with the menopausal depression group, the compound high-dose group has obviously decreased content of interleukin-2 in the serum (P<0.01); compared with the menopausal depression group, the compound medium-dose group, the compound low-dose group and the paroxetine group do not have any statistical significance in the content of the interleukin-2 in the serum (P<0.05); and with respect to IL-8 cytokine, no statistical significance is achieved in the medication groups.

TABLE 12
Influences of the drug on the content of IL-6, IL-2 and IL-8 in serum of rats

| Groups | Cases | IL-6 (ng/ml) | IL-2 (ng/L) | IL-8 (ng/L) |
|---|---|---|---|---|
| Compound | 6 | 15.1632 ± 2.5854** | 323.770 ± 25.210** | 190.713 ± 35.600 |
| high-dose | | | | |
| group | | | | |
| Compound | 6 | 15.8653 ± 3.5416** | 366.039 ± 34.878 | 198.928 ± 18.866 |
| medium-dose | | | | |
| group | | | | |
| Compound | 6 | 14.6433 ± 4.3287** | 404.937 ± 76.231 | 195.366 ± 30.335 |
| low-dose | | | | |
| group | | | | |
| Xiaoyao pill | 6 | 20.6554 ± 5.9550 | 430.547 ± 80.766 | 223.163 ± 17.476 |
| Paroxetine | 6 | 14.9800 ± 2.9183** | 341.853 ± 59.289* | 196.479 ± 14.596 |
| Sham-operated | 6 | 14.1132 ± 3.2655** | 358.383 ± 19.008 | 192.740 ± 36.210 |
| group | | | | |
| Menopause | 6 | 19.3443 ± 6.8291 | 386.076 ± 77.216 | 228.976 ± 21.495 |
| Menopausal | 6 | 25.8425 ± 4.9839 | 445.412 ± 41.193 | 234.050 ± 24.126 |
| depression | | | | |

In conclusion, compared with the rat model group of the mental depression model as well as the positive drugs of non-tricyclic western medicine of paroxetine and Xiaoyao pills, the compound drug group has obvious specific effects as follows: the compound drug group can effectively regulate: 1) the ratio of the content of the signal molecules cyclic adenosine monophosphate (CAMP) to cyclic guanosine monophosphate (CGMP); 2) regulate the ratio of content of the sex hormone dihydrotestosterone to the female hormone; 3) regulate the ratio of the follicle-stimulating hormone (FSH) to the oxytocin (OT); 4) regulate the ratio of the noradrenaline NA to the dopamine; and 5) decrease the content of the immune cytokines of interleukin-6 and interleukin-2 in serum of the depression model rats. The interleukin-2 is in a dose-effect relationship, the compound high-dose drug has an effect of decreasing the interleukin-2 in the serum, and the compound drug medium/low-dose groups do not have the statistical significance.

6. Possible Mechanisms of the Product in Treating the Menopausal Mental Depression:

By virtue of in-vivo adenylate cyclase-cyclic adenosine monophosphate (AC-cAMP) system functions, that is, by virtue of activation on a target receptor of an organism, the product can regulate the content ratio of the cAMP to the cyclic guanosine monophosphate cGMP in in-vivo serum, and perform positive-negative balanced regulation on the content of the influencing factors such as reproductive hormones including the follicle-stimulating hormone, the oxytocin and the dihydrotestosterone, adrenal hormones including the adrenocorticotrophic hormone ACTH and corticosterone Cor, neurotransmitters including the noradrenaline NA, 5-hydroxytryptamine 5-HT and dopamine, immune leukocyte factors including the interleukin-6 and interleukin-2, and the like. Since the compound drug group can effectively increase or decrease (or called positive-negative balanced regulation) the content of active markers secreted by the target receptors, such as internal secretion, neurotransmitters, sex hormones, immunity and the like, of the rats suffering from the mental depression, multiple neurocrine influencing factors are regulated from the disorder state to the healthy state. The compound drug is better than similar traditional Chinese medicines and some synthetic chemicals for treating the menopausal mental depression in the market.

The applicant is engaged in researches of single-flavor and compound drugs of a sea-buckthorn drug (sea buckthorn pulp oil) for a long time. Through basic and application researches in aspects of pharmacology, active ingredients, in-vivo biochemistry and nutrition metabolism, particularly a systematic research of pharmacology of a natural compound sea buckthorn pulp oil and total panax notoginseng saponins from stem and leave orthogonal design screening formula on action receptor bio-influencing factors, compared with curative effects of an “application of sea buckthorn pulp oil and sea buckthorn leaf extract in preparation of a drug for treating mental depression” with a patent number of ZL2005100962820, a composition compounded by the sea buckthorn pulp oil and the total panax notoginseng saponins from stem and leave has synergistic effects of reducing dose and strengthening effects, and the effects are more obvious and comprehensive.

On a basis of study on the sea buckthorn pulp oil, the applicant combines etiology and pathogenesis of the mental depression that the mental depression is related to clinical blood stasis and qi-stagnation in traditional Chinese medicine. In order to solve the pathogenesis in treating the mental depression disease, by adding a natural compound drug compounded by the total panax notoginseng saponins from stem and leave into a single-flavor sea buckthorn pulp oil drug, the inventor discovers that the disorder state of the content ratio of the endocrine hormones, neurotransmitters, immune cell cytokines and cyclic nucleotide signal molecules in serum of the model rats can be corrected and regulated to the normal and healthy state in a certain medication cycle by virtue of a compounded formula ratio and dose, thereby treating the menopause and mental depression diseases. Compared with curative effects of a previous patent ZL2005100962820 proposed by the applicant, in addition to achieving more comprehensive and remarkable pharmacodynamic actions on action target molecules, through the medication dose, the compound composition can regulate the content ratio of the adenylate cyclase-cyclic adenosine monophosphate cAMP to cyclic guanosine monophosphate cGMP in the menopause and mental depression diseases, and perform the positive-negative balanced regulation on the content of multiple influencing factors such as the reproductive hormones including the follicle-stimulating hormone, the oxytocin and the dihydrotestosterone, the adrenal hormones including the adrenocorticotrophic hormone ACTH and corticosterone Cor, the immune leukocyte factors including the interleukin-6, interleukin-8 and interleukin-2, and the like. The composition is a new discovery field that is not explored by the above patent ZL2005100962820.

So far, except the patent applied by the inventor, no literature reports treatment of the mental depression disease by using the drug compounded by the sea buckthorn pulp oil and the total panax notoginseng saponins from stem and leave in China and abroad. No ideal safe and simplified drug for preventing and treating the above diseases appears in a current market.

Limited to the number of words in documents, observation of clinical cases on the atypical depressive disorder should be provided as required at any time. The above embodiments are only used for describing technical concepts and characteristics of the present disclosure. A purpose of the present disclosure is to enable those skilled in the art to know and implement the content of the present disclosure, instead of limiting a protection scope of the present disclosure. All equivalent transformations or modifications made according to the spiritual essence of the present disclosure should be included in the protection scope of the present disclosure.

What is claimed is:

1. A natural pharmaceutical composition for treating menopausal mental depression and atypical depressive disorder, wherein the composition consists of following components in percentage by mass:
70%-97.5% of sea buckthorn pulp oil and 2.5%-30% of total panax notoginseng saponins from stem and leave, wherein the sea buckthorn pulp oil is extracted from fresh sea buckthorn fruits without seeds, and the total panax notoginseng saponins from stem and leave is extracted from the stems and leaves of panax notoginseng;
wherein a mass ratio of hexadecylenic acid to hexadecenoic acid contained in the sea buckthorn pulp oil is (1.02-1.20):1; and
a mass ratio of Rb3 to Rb1 contained in the total panax notoginseng saponins from stem and leave is 1:(0.16-0.20).

2. The natural pharmaceutical composition for treating menopausal mental depression and atypical depressive disorder according to claim 1, wherein
the composition consists of following components in percentage by mass:
90% of the sea buckthorn pulp oil and 10% of the total panax notoginseng saponins from stem and leave.

3. The natural pharmaceutical composition for treating menopausal mental depression and atypical depressive disorder according to claim 1, wherein
the composition consists of following components in percentage by mass: 96.7% of the sea buckthorn pulp oil and 5.3% of the total panax notoginseng saponins from stem and leave.

4. The natural pharmaceutical composition for treating menopausal mental depression and atypical depressive disorder according to claim 1, wherein
the composition consists of following components in percentage by mass: 84.0% of the sea buckthorn pulp oil and 16.0% of the total panax notoginseng saponins from stem and leave.

5. The natural pharmaceutical composition for treating menopausal mental depression and atypical depressive disorder according to claim 1, wherein
the composition consists of following components in percentage by mass: 92.7% of the sea buckthorn pulp oil and 7.3% of the total panax notoginseng saponins from stem and leave.

6. The natural pharmaceutical composition for treating menopausal mental depression and atypical depressive disorder according to claim 1, wherein
the composition consists of following components in percentage by mass: 70.0% of the sea buckthorn pulp oil and 9.0% of the total panax notoginseng saponins from stem and leave.

7. The natural pharmaceutical composition for treating menopausal mental depression and atypical depressive disorder according to claim 1, wherein the composition consists of following components in percentage by mass: 92.5% of the sea buckthorn pulp oil and 7.5% of the total panax notoginseng saponins from stem and leave.

8. The natural pharmaceutical composition for treating menopausal mental depression and atypical depressive disorder according to claim 1, wherein
the natural pharmaceutical composition is compounded by using the sea buckthorn pulp oil and the total panax notoginseng saponins from stem and leave as active ingredients and is prepared into soft capsules or oral liquid preparations.

9. An application of the natural pharmaceutical composition of claim 1 in preparation of a drug and/or a health care product for treating menopause, menopausal mental depression or atypical depressive disorder.

10. The application of the natural pharmaceutical composition in preparation of a drug and/or a health care product for treating menopause, menopausal mental depression or atypical depressive disorder according to claim 9, wherein
the drug and/or the health care product is a drug and/or health care product for regulating a content ratio of oxytocin, estradiol, dihydrotestosterone and related marker molecules in serum of the menopausal mental depression or the atypical depressive disorder.

11. The application of the natural pharmaceutical composition in preparation of a drug and/or a health care product for treating menopause, menopausal mental depression or atypical depressive disorder according to claim 9, wherein
the drug and/or the health care product is a drug and/or health care product for increasing a content ratio of cAMP/cGMP in the serum.

12. The application of the natural pharmaceutical composition in preparation of a drug and/or a health care product for treating menopause, menopausal mental depression or atypical depressive disorder according to claim 9, wherein
the drug and/or the health care product is a drug and/or health care product for increasing a content ratio of dihydrotestosterone to estradiol.

13. The application of the natural pharmaceutical composition in preparation of a drug and/or a health care product for treating menopause, menopausal mental depression or atypical depressive disorder according to claim 9, wherein
the drug and/or the health care product is a drug and/or health care product for decreasing a content of follicle-stimulating hormones and increasing a content of oxytocin.

14. The application of the natural pharmaceutical composition in preparation of a drug and/or a health care product for treating menopause, menopausal mental depression or atypical depressive disorder according to claim 9, wherein
the drug and/or the health care product is a drug and/or health care product for increasing a content ratio of noradrenaline and increasing a content ratio of dopamine to serotonin.

15. The application of the natural pharmaceutical composition in preparation of a drug and/or a health care product for treating menopause, menopausal mental depression or atypical depressive disorder according to claim 9, wherein
the drug and/or the health care product is a drug and/or health care product for decreasing cytokines contents of immune interleukin-6 and interleukin-2.
